(19) United States
(12) Patent Application Publication
(10) Pub. No.: US 2011/0065208 A1
(43) Pub. Date: Mar. 17, 2011

(54) DETERMINATION AND ESTIMATION OF ABSORPTION AND DISTRIBUTION OF SUBSTANCES IN BRAIN TISSUE

(75) Inventors: Hinnerk Boriss, Leipzig (DE); Johannes Schmitt, Leipzig (DE)

(73) Assignee: SOVICELL GMBH, Leipzig (DE)

(21) Appl. No.: 12/864,269

(22) PCT Filed: Jan. 26, 2009
(86) PCT No.: PCT/EP2009/000482

§ 371 Date: Oct. 27, 2010

(30) Foreign Application Priority Data
Jan. 25, 2008 (DE) 10 2008 007 673.2

(51) Int. Cl. G01N 33/92; G01N 33/53

(52) U.S. Cl. 436/501; 436/71

(57) ABSTRACT

A method determines or estimates the absorption and partition of substances into brain tissue and parameters derivable therefrom, such as the permeability of substances through the blood-brain barrier and also, more particularly, the proportion of substances freely available in the brain. The partition coefficient of the corresponding substance between an aqueous phase and the lipid phase is determined with the help of lipid membranes which are preferably contained in a test kit and immobilized on a solid body as a carrier material. The partition coefficient can be subsequently used as a parameter in a QSAR model to determine the blood-brain partition coefficient.

### RELATED APPLICATION

This is a §371 of International Application No. PCT/EP2009/000482, with an international filing date of Jan. 26, 2009 (WO 2009/092614 A1, published Jul. 30, 2009), which is based on German Patent Application No. 10 2008 007 673.2, filed Jan. 25, 2008, the subject matter of which is incorporated by reference.

### TECHNICAL FIELD

This disclosure relates primarily to methods for determining or estimating the absorption and partition of substances into brain tissue and parameters derivable therefrom such as permeability of substances through the blood-brain barrier and also, more particularly, the proportion of substances freely available in the brain. The logarithmized partition or distribution coefficient of the corresponding substance between blood or blood plasma on the one hand and brain on the other hand is preferably provided.

### BACKGROUND

Recognizing at an early stage and estimating the absorption behavior of substances in brain tissue are essential in the development of CNS-active substances and currently an intensive field of research and development. However, even for substances for which the site of action is not brain tissue, recognizing potential brain penetration (in this case, often undesired) at an early stage can be of crucial importance in the optimization of substance properties. In addition to various cell-based approaches, which are often based on primary cultures or specially modified cell lines and are hence fairly costly and inconvenient, in silico methods (“running on the computer”) above all are also used for estimating the blood plasma-brain partition. The appeal of these approaches is that predictions of the properties of the substances can even be made prior to substance synthesis. However, these approaches are often afflicted by a fairly low predict-ability (accuracy of prediction).

To determine or estimate the permeability or activity of the blood-brain barrier, use is often made of the blood-brain partition coefficient log BB, which is the logarithmized ratio of substance concentration in brain tissue and in blood or blood plasma. Accordingly, the following applies:

$$log BB = log ( \frac{{c}_{substance}^{brain}}{{c}_{substance}^{blood}} ) . \qquad ( 1 )$$

The permeability of the blood-brain barrier is determined either in vivo from pharmacokinetic measurements in animal models, which likewise provide the blood-brain partition coefficient, or in vitro by using primary cell cultures (an overview can be found in [U. Bickel, NeuroRx 2 (2005), 15-26]). A further approach makes use of in silico methods in which log BB is estimated from molecular parameters with the help of computer programs (“in silico”). [see U. Norinder, M. Haeberlein, Advanced Drug Delivery Reviews 543 (2002), 291-313].

The in vivo methods are generally fairly costly and inconvenient and not suitable for determining a large number of substances. An established methodology for estimating the permeability of the blood-brain barrier therefore makes use of purely in silico models which are based on molecular parameters. These methods are relatively fast and cost-effective, but often only provide an inadequate description of the partition between blood and brain.

Generally, to estimate the blood-brain partition coefficient log BB in silico, use is made of QSAR (quantitative structure-activity relationship) models which correlate the contributions of calculated or predefined molecular parameters of the corresponding substances in a relationship of the following general form:

log BB=ΣaᵢPᵢ+const. (2)

Here, aᵢ are the coefficients of the molecular parameters Pᵢ, which are obtained from multilinear regressions. Parameters which are often used are the octanol-water partition coefficients calculated here, the polar surface area PSA, the molar volume Vₘ and the molecular weight MW, and combinations of some or all parameters. Further approaches avail themselves of Abraham descriptors [e.g., J. A. Platts, M. H. Abraham, Y. H. Zhao, A. Hersey, L. Ijaz, D. Butina Eur. J. Med. Chem. 36 (2001), 719-730]. However, it is common to all approaches that they are based on such molecular parameters and do not explicitly consider the affinity of a substance for the constituents of blood.

The availability of a substance in the brain depends, however, not only on how much of it has passed into the brain but also on what proportion of the substance is freely available in the brain. This proportion is not least dependent on how much of the substance is non-specifically bound by proteins and lipids. However, estimating the free fraction in the brain is, to date, only possible in vivo or by costly and inconvenient in vitro dialysis against brain extract.

Accordingly, it could be helpful to provide an improved method for determining or estimating the absorption and partition of substances into brain tissue and also, more particularly, to determine or estimate the blood-brain partition coefficient log BB and the permeability of substances through the blood-brain barrier. This method should provide very high flexibility with regard to consideration of substance parameters and molecular parameters and, furthermore, be able to consider the binding of the corresponding substance to blood or blood plasma. Lastly, it should be able to routinely carry out key steps of such a method as easily as possible, for example, with the help of test kits.

### SUMMARY

We provide a method of determining or estimating absorption and partition of substances into brain tissue and parameters derivable therefrom including determining a partition coefficient of a corresponding substance between an aqueous phase and a lipid phase in conjunction with lipid membranes contained in a test kit and immobilized on a solid body as a carrier material.

We also provide a solid body including particles, wherein lipid membranes or lipid bilayers are immobilized on at least part of inner and outer surfaces of the solid body, and at least some of the lipids are brain tissue lipids originating from brain tissue extracts.

We further provide an aqueous suspension including the solid body.

We still further provide a kit for determining a partition coefficient of a substance between an aqueous phase and a lipid phase used for determining or estimating absorption and partition of substances into brain tissue including the solid body.

### DETAILED DESCRIPTION

We provide methods for determining or estimating the absorption and partition of substances into brain tissue. Such methods can serve, more particularly, both as a method for determining or estimating the permeability of substances through the blood-brain barrier and, more particularly, also for determining or estimating the proportion of substances freely available in the brain. Generally, it is common to these methods that the corresponding information is provided in the form of the preferably logarithmized partition coefficient of the substances between blood or blood plasma on the one hand and brain on the other hand. In the determination or estimation of the proportion of substances freely available in the brain, this information can also be provided, more particularly, as a percentage of the entire amount of this substance localized in the brain.

With the help of lipid membranes, more particularly with the help of lipid bilayers immobilized on a solid body as a carrier material, the partition coefficient log MA_{brain} of the corresponding substance between an aqueous phase and the lipid phase (the immobilized lipid membranes) is initially determined (this partition coefficient can also be referred to as membrane affinity). The lipid membranes or lipid bilayers are preferably contained in a test kit, as will be explained later in more detail.

The partition coefficient log MA_{brain} thus obtained (or the membrane affinity thus obtained) can then be used in more preferred aspects of the method as a parameter in a QSAR model (QSAR stands for “quantitative structure-binding relationship”) which is intended to determine the blood-brain partition coefficient.

When determining or estimating the proportion of substances freely available in the brain (fu_{brain}), it is even possible to recover this proportion almost directly from the partition coefficient log MA_{brain} obtained (the membrane affinity). To this end, it is possible to carry out a linear regression, for example, according to the formula “fu_{brain}=10exp(A*log MA_{brain}+E)”, in which the values recovered for the partition coefficient log MA_{brain} can be used.

“Aqueous phase” shall be understood to mean a phase which works quite overwhelmingly with water as a solvent or a dispersant. This means that, generally, only minimal proportions of organic solvent or organic dispersant are present in the corresponding phase. The corresponding aqueous phase is generally buffered, i.e., adjusted to a particular pH.

The lipids of the lipid membrane or the lipid bilayer can, in principle, be from a very diverse range of lipids or lipid mixes. Thus, membranes of purely phosphatidylcholine (PC) may be used. Likewise, use may be made of membranes of phosphatidylcholine (PC) having a 20% proportion of the negatively charged lipid phosphatidylserine (PS), wherein this 20% proportion corresponds to the concentration of PS in brain tissue.

However, it is preferred that the lipid of the lipid membrane is at least one brain tissue lipid or a mixture of such brain tissue lipids. In turn, it is preferred that lipid extracts from brain tissue are provided for the corresponding lipid membranes or lipid bilayers.

The partition coefficient (or the membrane affinity) of the corresponding substance between an aqueous phase and the lipid phase can be used as a parameter in a QSAR model for the blood-brain partition coefficient. It is advantageous in this case when, in addition to this (experimentally) determined parameter, further parameters, which can also be further experimentally determined parameters, enter the QSAR model.

With regard to such parameters, a further parameter can be, for example, the substance parameter molar volume (Vₘₒₗ), which is obtainable for the corresponding substance by a comparatively simple calculation. An even more preferred further parameter can be the substance parameter of molecular weight (MW), which can generally be easily calculated from the composition of the substance.

A further, comparatively easily obtainable substance parameter for consideration in the QSAR model is the value of the “polar surface area” (PSA), which can likewise be easily calculated (see, for example, P. Ertl, B. Rhode, P. Selzer J. Med. Chem. 43 (2000) 3714-3717).

Lastly, the additional parameter of binding of the corresponding substance to blood or blood plasma is of particular importance. Thus, in addition to the substance parameters mentioned (molecular parameters), the affinity of a substance for blood is also considered. This affinity, of course, also plays an important role in the crossing of this substance from blood into the brain through the blood-brain barrier.

Binding of the corresponding substance to blood or blood plasma is preferably understood as the logarithmized ratio of the bound fraction of this substance in blood/blood plasma to the free fraction of this substance in blood/blood plasma.

Generally, binding of the corresponding substance to blood/blood plasma can be determined and taken into consideration in terms of binding the substance to one or more blood/plasma proteins. The plasma proteins HSA (human serum albumin) and/or AGP (α₁-acid glycoprotein) are preferably considered. Further preferably, the binding of the substance to human or animal whole plasma can also be considered.

The last mentioned parameters, binding of the substance to one or more blood/plasma proteins and also binding of the substance to human or animal whole plasma, can also, more particularly, be easily determined experimentally, which is illustrated, for instance, in working examples 5 and 6 (see below).

As will be apparent from the observations heretofore, it is advantageous when, in addition to the (experimentally) determined partition coefficient of the substance between an aqueous phase and the lipid phase, at least one of the preferred parameters mentioned (MW, PSA, binding of the substance in blood) is taken into consideration, preferably all three of these parameters.

Accordingly, more preferably, the corresponding QSAR model for the blood-brain or blood plasma-brain partition coefficient is represented by the following formula:

log BB=A*log MA_{brain}+B*PSA+C*MW+D*log K^{B/F}+E. (3)

The corresponding terms and parameters are explained as follows:
log BB Logarithmized blood-brain partition coefficient
log MA_{brain} Logarithmized partition coefficient of the corresponding substance between aqueous phase and lipid phase
PSA Polar surface area
MW Molecular weight
log K^{B/F} Logarithmized ratio of bound (B) to free (F) fraction of the corresponding substance in blood/blood plasma.

It is known that it is also possible to use further molecular parameters or molecular properties in the abovementioned QSAR relationship.

Similarly, when determining or estimating the proportion of freely available substances in the brain, it may be preferred that, in addition to the experimentally recoverable parameter log MA_{brain}, other parameters such as, for example, log K^{B/F} or the abovementioned parameters determinable by calculation (MW, PSA or the like) also play a role. These parameters can then be taken into consideration, for example, as interference factors, for example, while carrying out a linear regression according to the above formula “fu_{brain}=10exp(A*log MA_{brain}+E)”. Thus, preferably, in determining the proportion of freely available substances in the brain, the molecular weight of the corresponding substance and/or the PSA (polar surface area) value of the corresponding substance and/or the binding of the corresponding substance to blood or blood plasma are to be considered as interference or correction factors, wherein, for the latter, the binding of the corresponding substance to blood or blood plasma is more preferably represented by binding this substance to the plasma proteins HSA (human serum albumin) and/or AGP (α₁-acid glycoprotein) and/or by binding this substance to human or animal whole plasma.

We further provide a solid body which is present, more particularly, in the form of particles, wherein lipid membranes or lipid bilayers are immobilized on at least part of the inner and outer surfaces of this solid body. A feature of this solid body is that at least some, but preferably all, of the lipids of the lipid membranes or lipid bilayers are brain tissue lipids, i.e., such lipids as occur in the brain tissue of animals or also of humans. The brain tissue lipids mentioned preferably originate from brain tissue extracts. The corresponding lipids and extracts are known and may be obtained commercially from various companies.

The solid bodies can also be described such that the lipid membranes or lipid bilayers are applied to the actual solid body as a carrier material and immobilized there. The production of such modified and coated solid bodies is known and described in detail, for example, in DE-A1-100 48 822.

It must be emphasized that the solid bodies are insoluble in aqueous solution. They can be made of organic or even inorganic material, more particularly silicates, aluminates, borates, or zeolites for the latter. Metals and noble metals are also usable.

The solid bodies are present, more particularly, in the form of particles. In this form, the surface area of the solid body is considerably increased. The solid body, more particularly the particles, may be nonporous, but preferably also porous, which is likewise associated with a further enlargement in surface area.

As a result, preferred solid bodies or particles on which the lipid membranes or lipid bilayers from brain tissue lipids are immobilized, are present in the form of beads.

We further provide a suspension, preferably an aqueous suspension, which contains the described solid body or the particles having a lipid membrane/lipid bilayer from brain tissue lipids.

Providing the solid body or the particles in the form of a suspension facilitates their use, more particularly in the method.

Lastly, we provide a kit for determining the partition coefficient of a substance between an aqueous phase and a lipid phase (or else the membrane affinity), wherein the partition coefficient thus obtained is preferably intended to determine or estimate absorption and partition of substances into brain tissue, more particularly within the context of our methods. This kit comprises the solid body or the particles and/or the suspension.

Accordingly, in such a kit, the particles (beads) coated with the brain tissue lipids can be provided as a suspension in an aqueous solvent. This aqueous system is generally a buffer system, such as, for example, PBS (phosphate-buffered saline, namely 10 mM phosphate buffer with 155 mM NaCl, pH 7.4). This suspension can already be present in the wells of a microtiter plate and, if appropriate, distributed at the necessary concentrations. Such microtiter plates prepared in this way can be present in a frozen state, in which case they can be transported and stored in this state. These plates are then thawed immediately prior to use of the kit thus obtained.

Using the kit, the partition coefficient of the substances to be studied between the aqueous phase (buffer) and the lipid phase (beads) is then experimentally determined, as described in connection with the method wherein, optionally, a fully automated apparatus can be used.

The features described result from the following description of preferred examples. The various features can each be realized alone or in combination with one another.

### 1. Providing a Suitable Carrier Material

5 g of a porous particulate silicate material (Nucleoprep 4000-12 from Macherey-Nagel, Germany) are added to a silane solution consisting of 1.05 mL of N-(2-aminoethyl)-3-aminopropyltrimethoxysilane (EDA) and 27 μL of concentrated acetic acid in 100 mL of deionized water and stirred slowly for three hours. Afterwards, the silicate material is sedimented, washed three times with deionized water, and dried at 80° C.

Subsequently, 5 g of the material thus produced are added to a solution of 5 g of epoxypropanol in 50 mL of ethanol and stirred overnight (16 hours). Afterwards, the material is sedimented, washed three times with ethanol, washed three times with deionized water, and dried at 80° C.

2. Producing the Carrier Material with Immobilized Lipid Membrane (as a Suspension)

440 mg of a brain lipid extract (Avanti Polar Lipids Inc., USA) are dissolved in 50 mL of chloroform. This solution is evaporated under reduced pressure in a glass flask in a rotating rotary evaporator, wherein a thin lipid film deposits on the wall.

The lipid film is swollen overnight in a water vapor atmosphere; subsequently, 88 mL of a buffer (20 mM HEPES, pH 7.0, 30 mM NaCl, 2 mM CaCl₂) are added, and the lipid film is vesiculated by means of a high-pressure homogenizer.

5 g of the carrier material provided above are added to the vesicle solution thus produced and incubated overnight. Excess lipid is removed by washing three times with buffer (20 mM HEPES, pH 7.0, 30 mM NaCl, 2 mM CaCl₂). Finally, this is followed by rebuffering in PBS (phosphate-buffered saline, 10 mM sodium phosphate, 0.9% NaCl, pH 7.4) and filling up to 25 mL with buffer. The suspension obtained is then used to determine the brain lipid-water partition coefficient, as described below.

3. Determining the Brain Lipid-Water Partition Coefficient log MA_{brain} of trifluoperazine

To determine the brain lipid-water partition coefficient, a total of 5 wells of a 96-well microtiter plate are filled as follows:
Well 1: 405 μL PBS
Well 2: 375 μL PBS+30 μL suspension (see 2.)
Well 3: 357 μL PBS+48 μL suspension (see 2.)
Well 4: 338 μL PBS+77 μL suspension (see 2.)
Well 5: 405 μL PBS.

To each of these wells, 45 μL of a 500 μM solution of trifluoperazine in PBS/10% DMSO are added, and after mixing by resuspension, the solid body is separated from the supernatant by sedimentation. The concentration of the substance in the supernatant is quantified by UV measurement, and the brain lipid-water partition coefficient log MA_{brain} is calculated through the following equation:

$$log MA = log ( \frac{{V}_{total}}{{V}_{lipid}} \cdot \frac{{c}_{total} - {c}_{water}}{{c}_{water}} ) . \qquad ( 4 )$$

In the equation are:
Vₜₒₜₐₗ Total volume per well
V_{lipid} Lipid volume per well
cₜₒₜₐₗ Substance concentration of wells 1 and 5 without binding partner
c_{water} Substance concentration in the supernatant of wells 2-4 with binding partner.

A brain lipid-water partition coefficient of log MA_{brain}=4.7 is obtained.

### 4. Determining the Brain Lipid-Water Partition Coefficient log MA_{brain} for Further Substances

To determine the brain lipid-water partition coefficient, two 96-well microtiter plates are filled row by row as follows:
Row 1: 405 μL PBS
Row 2: 375 μL PBS+30 μL suspension (see 2.)
Row 3: 357 μL PBS+48 μL suspension (see 2.)
Row 4: 338 μL PBS+77 μL suspension (see 2.)
Row 5: 285 μL PBS+120 μL suspension (see 2.)
Row 6: 210 μL PBS+195 μL suspension (see 2.)
Row 7: 105 μL PBS+300 μL suspension (see 2.)
Row 8: 405 μL PBS.

A broad binding range is covered by this filling of the plates. To each of these prefilled plates, 45 μL of a 500 μM solution of one of the following substances in PBS/10% DMSO are added row by row: acetylsalicylic acid, chlorpromazine, indomethacin, salicylic acid, verapamil, phenylbutazone, cimetidine, promazine, propranolol, clonidine, atenolol, caffeine, desipramine, ibuprofen, imipramine, quinidine, theophylline, trifluoperazine, and antipyrine. After mixing by resuspension, the solid body is separated from the supernatant by sedimentation.

The concentration of the substance in the supernatant is quantified by HPLC, and the brain lipid-water partition coefficient log MA_{brain} is calculated through the abovementioned equation (4), wherein, in this case, Vₜₒₜₐₗ is the total volume per well, V_{lipid} is the lipid volume per well, cₜₒₜₐₗ is the substance concentration of rows 1 and 8 without binding partner, and c_{water} is the substance concentration in the supernatant of wells 2-7 with binding partner.

Table 1 shows the results of the measurements:

TABLE 1
Brain lipid-water partition coefficients obtained

| Substance | logMA_{brain} |
|---|---|
| Acetylsalicylic acid | 1.40 |
| Chlorpromazine | 3.70 |
| Indomethacin | 2.30 |
| Salicylic acid | 1.30 |
| Verapamil | 2.36 |
| Phenylbutazone | 1.29 |
| Cimetidine | 1.80 |
| Promazine | 3.20 |
| Propranolol | 2.70 |
| Clonidine | 1.30 |
| Atenolol | 0.90 |
| Caffeine | 1.36 |
| Desipramine | 3.10 |
| Ibuprofen | 1.70 |
| Imipramine | 2.84 |
| Quinidine | 2.10 |
| Theophylline | 1.34 |
| Trifluoperazine | 4.70 |
| Antipyrine | 1.33 |

### 5. Determining HSA Binding Constants K_{d}^{HSA}

To each prefilled HSA binding plate (TRANSIL® HSA Binding Kit; NIMBUS, Germany), 45 μL of a 500 μM solution of one of the following substances in PBS/10% DMSO are added row by row: acetylsalicylic acid, chlorpromazine, indomethacin, salicylic acid, verapamil, phenylbutazone, cimetidine, promazine, propranolol, clonidine, atenolol, caffeine, desipramine, ibuprofen, imipramine, quinidine, theophylline, trifluoperazine, and antipyrine. After mixing by resuspension, the solid body is separated from the supernatant by sedimentation. The concentration of the substance in the supernatant is quantified by HPLC, and the dissociation constants K_{d}^{HSA} are calculated through the following equation:

$${K}_{D} = {[ drug ]}_{free} \cdot \frac{1 - \frac{{[ drug ]}_{bound}}{[ protein ]}}{\frac{{[ drug ]}_{bound}}{[ protein ]}} . \qquad ( 5 )$$

In the equation are:
[drug]_{bound}: Concentration of bound substance
[drug]_{free}: Concentration of free substance in the supernatant
[protein]: Protein concentration in the respective wells.

The following values are obtained:

TABLE 2
K_{D}^{HSA} values obtained

| Substance | K_{D}^{HSA} [mol/L] |
|---|---|
| Acetylsalicylic acid | 3.6E−04 |
| Chlorpromazine | 2.1E−05 |
| Indomethacin | 6.4E−07 |
| Salicylic acid | 2.5E−05 |
| Verapamil | 5.0E−04 |
| Phenylbutazone | 5.4E−06 |
| Cimetidine | 9.2E−04 |
| Promazine | 9.9E−05 |
| Propranolol | 6.1E−04 |
| Clonidine | 1.9E−03 |
| Atenolol | 2.4E−03 |
| Caffeine | 1.5E−03 |
| Desipramine | 2.0E−04 |
| Ibuprofen | 4.2E−07 |
| Imipramine | 2.2E−04 |
| Quinidine | 4.7E−04 |
| Theophylline | 5.4E−03 |
| Trifluoperazine | 1.2E−05 |
| Antipyrine | 2.7E−03 |

### 6. Determining AGP Binding Constants K_{d}^{AGP}

To each prefilled AGP binding plate (TRANSIL® AGP Binding Kit; NIMBUS, Germany), 45 μL of a 50 μM solution of one of the following substances in PBS/10% DMSO are added row by row: acetylsalicylic acid, chlorpromazine, indomethacin, salicylic acid, verapamil, phenylbutazone, cimetidine, promazine, propranolol, clonidine, atenolol, caffeine, desipramine, ibuprofen, imipramine, quinidine, theophylline, trifluoperazine, and antipyrine. After mixing by resuspension, the solid body is separated from the supernatant by sedimentation. The concentration of the substance in the supernatant is quantified by HPLC, and the dissociation constants Kd^{AGP} are calculated through the equation (5) already mentioned above:

$${K}_{D} = {[ drug ]}_{free} \cdot \frac{1 - \frac{{[ drug ]}_{bound}}{[ protein ]}}{\frac{{[ drug ]}_{bound}}{[ protein ]}} . \qquad ( 5 )$$

In the equation are:
[drug]_{bound}: Concentration of bound substance
[drug]_{free}: Concentration of free substance in the supernatant
[protein]: Protein concentration in the respective wells.

The following values are obtained:

TABLE 3
K_{D}^{AGP} values obtained

| Substance | K_{D}^{AGP} [mol/L] |
|---|---|
| Acetylsalicylic acid | 4.6E−04 |
| Chlorpromazine | 7.2E−07 |
| Indomethacin | 4.3E−05 |
| Salicylic acid | 6.4E−04 |
| Verapamil | 4.9E−06 |
| Phenylbutazone | 7.0E−05 |
| Cimetidine | 2.5E−04 |
| Promazine | 1.6E−06 |
| Propranolol | 5.4E−06 |
| Clonidine | 6.9E−05 |
| Atenolol | 5.8E−04 |
| Caffeine | 5.8E−04 |
| Desipramine | 5.1E−06 |
| Ibuprofen | 6.7E−04 |
| Imipramine | 3.1E−06 |
| Quinidine | 1.3E−05 |
| Theophylline | 8.1E−04 |
| Trifluoperazine | 2.5E−07 |
| Antipyrine | 3.8E−04 |

7. Determining a QSAR by Multilinear Correlation of Plasma-Brain Partition Coefficient log BB with log MA_{brain}, PSA (Polar Surface Area), Molecular Weight MW, and log K^{B/F}

To estimate the plasma-brain partition coefficients, a multilinear regression was carried out according to the equation:

log BB=A*log MA_{brain}+B*PSA+C*MW+D*log K^{B/F}+E. (6)

The log K^{B/F} value represents the logarithmized ratio of the bound fraction to the free fraction in blood plasma, which has been approximately calculated from the dissociation constants K_{D}^{AGP} and K_{D}^{HSA} according to:

$$\begin{matrix}log {K}^{B / F} = log ( \frac{1 - {f}_{u}}{{f}_{u}} ) & ( 7 ) \\ {f}_{u} = \frac{1}{1 + [ HSA ] / {K}_{d}^{HSA} + [ AGP ] / {K}_{d}^{AGP}} & ( 8 )\end{matrix}$$

fᵤ Free substance concentration in plasma
[drug]ₜₒₜₐₗ^{plasma} Total substance concentration in plasma
[HSA] HSA concentration in blood plasma, 588.2 μM
[AGP] AGP concentration in blood plasma, 24.4 μM
K_{d}^{HSA} Dissociation constant of the substance—HSA complex
K_{d}^{AGP} Dissociation constant of the substance—AGP complex.

The parameters used are summarized in Table 4.

TABLE 4
Parameter set for determining the LFER

| Test set | logBB | logMA_{brain} | K_{D}^{HSA} [mol/L] | K_{D}^{AGP} [mol/L] | logK^{B/F} | MW | PSA |
|---|---|---|---|---|---|---|---|
| Acetylsalicylic acid | −0.5 | 1.40 | 3.6E−04 | 4.6E−04 | 0.23 | 180.2 | 63.6 |
| Chlorpromazine | 1.06 | 3.70 | 2.1E−05 | 7.2E−07 | 1.79 | 318.9 | 31.8 |
| Indomethacin | −1.26 | 2.30 | 6.4E−07 | 4.3E−05 | 2.97 | 357.8 | 68.5 |
| Salicylic acid | −1.1 | 1.30 | 2.5E−05 | 6.4E−04 | 1.37 | 138.1 | 57.5 |
| Verapamil | −0.7 | 2.36 | 5.0E−04 | 4.9E−06 | 0.79 | 454.6 | 64.0 |
| Phenylbutazone | −0.52 | 1.29 | 5.4E−06 | 7.0E−05 | 2.04 | 308.4 | 40.6 |
| Cimetidine | −1.42 | 1.80 | 9.2E−04 | 2.5E−04 | −0.13 | 252.3 | 114.2 |
| Promazine | 1.23 | 3.20 | 9.9E−05 | 1.6E−06 | 1.33 | 284.4 | 31.8 |
| Propranolol | 0.64 | 2.70 | 6.1E−04 | 5.4E−06 | 0.74 | 259.3 | 41.5 |
| Clonidine | 0.11 | 1.30 | 1.9E−03 | 6.9E−05 | −0.17 | 230.1 | 36.4 |
| Atenolol | −1.42 | 0.90 | 2.4E−03 | 5.8E−04 | −0.55 | 266.3 | 84.6 |
| Caffeine | −0.06 | 1.36 | 1.5E−03 | 5.8E−04 | −0.36 | 194.2 | 58.4 |
| Desipramine | 1.2 | 3.10 | 2.0E−04 | 5.1E−06 | 0.89 | 266.4 | 15.3 |
| Ibuprofen | −0.18 | 1.70 | 4.2E−07 | 6.7E−04 | 3.14 | 206.3 | 37.3 |
| Imipramine | 0.83 | 2.84 | 2.2E−04 | 3.1E−06 | 1.02 | 280.4 | 6.5 |
| Quinidine | −0.46 | 2.10 | 4.7E−04 | 1.3E−05 | 0.50 | 324.4 | 45.6 |
| Theophylline | −0.29 | 1.34 | 5.4E−03 | 8.1E−04 | −0.86 | 180.2 | 69.3 |
| Trifluoperazine | 1.44 | 4.70 | 1.2E−05 | 2.5E−07 | 2.17 | 407.5 | 35.0 |
| Antipyrine | −0.1 | 1.33 | 2.7E−03 | 3.8E−04 | −0.54 | 188.2 | 23.6 |

The following relationship is obtained:

log BB=0.782*log MA_{brain}−0.021*PSA−0.003*MW−0.201*log K^{B/F}+0.203. (9)

In this case, the coefficient of determination R² is 0.92.

### 8. Estimating the Plasma-Brain Partition Coefficients log BB of Substances of a Validation Set

To validate the relationship obtained in 7, the log MA_{brain} and log K^{B/F} of the substances ranitidine, amitryptilline, carbamazepine, lidocaine, procaine, mianserin, diphenhydramine, fluoxetine, tolbutamide were determined according to points 4.-6. and the log BB value was estimated together with the PSA (polar surface area), which was obtained from the literature, and the molecular weight MW by means of equation (9) in 7. Table 5 shows the results obtained.

TABLE 5
Comparison of the logBB_{calc} values calculated by means
of equation (9) with logBB values from the literature

| Test set | logBB_calc | logBB |
|---|---|---|
| Ranitidine | −1.17 | −1.23 |
| Amitryptilline | 1.38 | 0.87 |
| Carbamazepine | −0.09 | 0 |
| Lidocaine | −0.08 | 0.34 |
| Procaine | −0.41 | 0.05 |
| Mianserin | 1.47 | 0.99 |
| Diphenhydramine | 0.82 | 1.26 |
| Fluoxetine | 1.29 | 1.08 |
| Tolbutamide | −0.85 | −1.01 |

9. Determining the Free Fraction in the Brain fu_{brain} with the membrane affinity log MA_{brain}

To estimate the free fraction in the brain, a linear regression was carried out according to the equation

“fu_{brain}=10 exp(A*log MA_{brain}+E)”.

The parameters used are summarized in Table 6.

TABLE 6
Parameter set for determining the free fraction in brain

| Substance | logMA_brain | fu_brain |
|---|---|---|
| Chlorpromazine | 3.70 | 0.002 |
| Caffeine | 1.36 | 0.52 |
| Trifluoperazine | 4.70 | 0.0007 |
| Amitryptilline | 2.94 | 0.009 |
| Carbamazepine | 1.79 | 0.1185 |
| Fluoxetine | 3.52 | 0.004 |
| Phenytoin | 2.02 | 0.082 |
| Thioridazine | 3.74 | 0.001 |
| Haloperidol | 2.7 | 0.011 |

The following relationship is obtained:

fu_{brain}=−0.958*log MA_{brain}+0.805.

1. A method of determining or estimating absorption and partition of substances into brain tissue and parameters derivable therefrom comprising determining a partition coefficient of a corresponding substance between an aqueous phase and a lipid phase in conjunction with lipid membranes contained in a test kit and immobilized on a solid body as a carrier material.

2. The method as claimed in claim 1, wherein the absorption and partition of substances are determined or estimated by determining a logarithmized partition coefficient of the substances between blood or blood plasma and brain by using a partition coefficient determined with lipid membranes immobilized on a solid body as a parameter in a QSAR (quantitative structure-activity relationship) model for the blood-brain or blood plasma-brain partition coefficient.

3. The method as claimed in claim 1, wherein the lipid of the lipid membrane is at least one brain tissue lipid.

4. The method as claimed in claim 2, wherein the QSAR model for the blood-brain or blood plasma-brain partition coefficient considers molecular weight of a corresponding substance as a further parameter.

5. The method as claimed in claim 2, wherein the QSAR model for the blood-brain or blood plasma-brain partition coefficient considers a PSA (polar surface area) value of a corresponding substance as a further parameter.

6. The method as claimed in claim 2, wherein the QSAR model for the blood-brain or blood plasma-brain partition coefficient considers binding of a corresponding substance to blood or blood plasma as a logarithmized ratio of a bound fraction of the substance to a free fraction of the substance in blood or blood plasma.

7. The method as claimed in claim 6, wherein binding of the corresponding substance to blood or blood plasma is represented by binding of the substance to plasma proteins HSA (human serum albumin) and/or AGP (α₁-acid glycoprotein).

8. The method as claimed in claim 6, wherein binding of the corresponding substance to blood or blood plasma is represented by binding of the substance to human or animal whole plasma.

9. The method as claimed in claim 4, wherein the QSAR model for the blood-brain or blood plasma-brain partition coefficient considers at least two parameters.

10. The method as claimed in claim 9, wherein the QSAR model for the blood-brain or blood plasma-brain partition coefficient is represented by the following formula:
log BB=A*log MA_{brain}+B*PSA+C*MW+D*log K^{B/F}+E.

11. A solid body comprising particles, wherein lipid membranes or lipid bilayers are immobilized on at least part of inner and outer surfaces of the solid body, and at least some of the lipids are brain tissue lipids originating from brain tissue extracts.

12. An aqueous suspension comprising the solid body as claimed in claim 11.

13. A kit for determining a partition coefficient of a substance between an aqueous phase and a lipid phase used for determining or estimating absorption and partition of substances into brain tissue comprising the solid body as claimed in claim 11.

14. The method as claimed in claim 1, which determines or estimate permeability of substances through the blood-brain barrier.

15. The method as claimed in claim 1, which determines or estimates a proportion of substances freely available in the brain.

16. A kit for determining a partition coefficient of a substance between an aqueous phase and a lipid phase used for determining or estimating absorption and partition of substances into brain tissue comprising the solid body as claimed in claim 12.
